(19) United States
(12) Patent Application Publication
(10) Pub. No.: US 2004/0254148 A1
(43) Pub. Date: Dec. 16, 2004

(54) Pharmaceutical composition effective in treatment of mechanical allodynia, screening method of potential compound as said pharmaceutical composition, inspection method of mechanical allodynia, and treatment method of mechanical allodynia

(76) Inventors: Masanori Hizue, Aichi-ken (JP); Masayuki Yokoyama, Aichi-ken (JP)

Correspondence Address: WARNER-LAMBERT COMPANY, 2800 PLYMOUTH RD, ANN ARBOR, MI, 48105, US

(21) Appl. No.: 10/812,479

(22) Filed: Mar. 30, 2004

Related U.S. Application Data
(60) Provisional application No. 60/459,479, filed on Apr. 1, 2003.

(51) Int. Cl. A61K 31/675; A61K 31/66; A61K 31/4709; A61K 31/496

(52) U.S. Cl. 514/85; 514/114; 514/255.01; 514/310; 514/649

(57) ABSTRACT

The present invention aims to identify the molecule involved in mechanical allodynia, and to develop and provide a pharmaceutical composition to target this molecule such that the composition is effective in the treatment of mechanical allodynia. The present invention also aims to provide a screening method for compounds for use in such pharmaceutical compositions, a method of determining mechanical allodynia, and a treatment method of mechanical allodynia.

This application is a United States Utility application, which claims the benefit of priority to U.S. Provisional application Ser. No. 60/459,479 filed Apr. 1, 2003.

### BACKGROUND OF THE INVENTION

1. Field of the Invention

The present invention relates to a pharmaceutical composition effective in the treatment of mechanical allodynia. The present invention also relates to methods of identifying compounds for use in the treatment of mechanical allodynia. The present invention additionally relates to the use of compounds for the treatment of mechanical allodynia. The present invention further relates to a method of detection of mechanical allodynia. Also contemplated by the present invention is a treatment method for mechanical allodynia.

2. Description of the Related Art

There is a sickness known as neuropathic pain. Neuropathic pain is a pain arising from various types of neurological disorder, and includes the condition of allodynia. Allodynia is a state where pain is felt even as a consequence of stimulation that would not ordinarily cause pain. Allodynia includes chemical allodynia and mechanical allodynia. Chemical allodynia is allodynia developed after sensitization following contact with chemical agents, the cause being chemical action. Mechanical allodynia is allodynia developed as a result of physical trauma.

The NMDA (N-Methyl-D-Aspartate) receptor is a multi-subunit assembly. An NMDA (N-Methyl-D-Aspartate) receptor comprises two subunits; namely, NR1(ζ) and NR2(ε), and the ε subunit is further classified into four subunits of ε1 to ε4(NR2D). The combination of subunits ζ and ε is required for the NMDA receptor channel to demonstrate channel activity. The molecular diversity of the NMDA receptor channel is considered to be determined by the ε subunit (Nakanishi and Masu, Annu. Rev. Biophys. Biomol. Struct. 23 (1994), 319-348., Hollmann and Heinemann, Annu. Rev. Neurosci. 17 (1994), pp. 31-108□McBain and Mayer, Physiolog Rev 74: (1994) 723-760.).

The NMDA receptor protein has been reported to be involved in the process of chemical allodynia. It has been reported that the hyperalgesia caused by prostaglandin E2 (PGE2) disappears in mice where the genes for subunits ε1 and ε4(NR2D) have been knocked out, and, while the allodynia caused by PGE2 disappears in a knockout mouse for subunit ε1, it does not disappear in a knockout mouse for subunit ε4(NR2D) (Minami et al., Br J Phannacol. April 1997; 120(8): 1522-1526., Minami et al., Eur J Neurosci. June 1999; 11(6):1849-1856.). It has also been reported that while allodynia caused by PGF2α disappears in a knockout mouse for subunit ε4(NR2D), it does not disappear in a knockout mouse for subunit ε1 (Minamiet et al., Eur J Neurosci. June 1999; 11(6): 1849-1856.). It has been further shown that even if the symptom is the same, for example chemical allodynia, depending on the type of causal compounds, the molecule relating to the development of allodynia will differ.

However, it is still unclear as to what molecule is involved in the development of mechanical allodynia.

### SUMMARY OF THE INVENTION

It is the object of the present invention to identify the receptor protein molecule involved in mechanical allodynia. It is a further object of the present invention to provide a method of screening for a compound capable of binding to the receptor protein molecule. It is still further an object of the invention to provide a pharmaceutical composition comprising a compound effective in the treatment of mechanical allodynia. It is a further object of the present invention to provide a method for the detection of mechanical allodynia. It is also an object of the present invention to provide a method of treatment for mechanical allodynia.

The present inventors have shown that it is possible to control the development of mechanical allodynia as can be induced by the partial damage to the sciatic nerve in a mouse by knocking out the gene coding for subunit ε4(NR2D) of the NMDA receptor protein. This demonstrates that the NMDA ε4(NR2D) subunit has a central role in the development of mechanical allodynia. Therefore, a compound for inhibiting the NMDA ε4(NR2D) receptor protein function may be used as a pharmaceutical for the treatment or prevention of mechanical allodynia. Also the measurement of the function or expression levels of NMDA ε4(NR2D) receptor protein may be utilized as an index for the detection of mechanical allodynia.

The present invention provides the following numbered aspects:

1. Use of a compound which inhibits the function of an NMDA ε4(NR2D) receptor protein for the manufacture of a medicament for the treatment of mechanical allodynia.

2. Use of a compound according to aspect 1, wherein the compound for inhibiting the function of an NMDA ε4(NR2D) receptor protein is an antagonist of the NMDA ε4(NR2D) receptor protein.

3. Use of a compound according to according to aspect 2, wherein the antagonist is selected from the group consisting of (±)-4-(4-phenylbenzoyl) piperazine-2,3-dicarboxylic acid (PBPD); (R,E)-4-(3-phosphonoprop-2-enyl)piperazine-2-carboxylic acid (D-CPPene); (±)-6-(1H-Tetrazol-5-ylmethyl)decahydroisoquinoline-3-carboxylic acid (LY23353); α-Amino-5-(phosphonomethyl)[1,1′-biphenyl]-3-propanoic acid (EAB515); cis-4-(phosphonomethyl)piperidine-2-carboxylic acid (CGS 19755 ); D,L-(E)-2-amino-4-propyl-5-phosphono-3-pentenoic acid (CGP 39653); tanshinone IIA; tanshinone IIB; 2-(3-methylphenyl)-2-adamantanemethanamine (CEB-1604); N1,N4,N8-tri-benzyl-spermidine (TB-3-4); and Memantine.

4. A pharmaceutical composition for treating mechanical allodynia comprising a compound as defined in any one of aspects 1 to 3 and a pharmaceutically acceptable diluent or carrier.

5. Use of a pharmaceutical product according to aspect 4 in the manufacture of a medicament for the treatment of mechanical allodynia

6. A method of screening for a compound which binds to NMDA ε4(NR2D), the method comprising the following steps (a) to (c):

(a) contacting NMDA ε4(NR2D) receptor protein with a test compound;

(b) detecting the binding of the test compound to NMDA ε4(NR2D) receptor protein; and

(c) selecting a test compound that binds with the NMDA ε4(NR2D) receptor protein.

7. A method of screening for a compound which binds to NMDA ε4(NR2D), the method comprising the following steps (a) to (c):

(a) contacting a test compound with a cell that expresses an NMDA ε4(NR2D) receptor gene in the presence of a ligand of the NMDA ε4(NR2D) receptor protein;

(b) detecting the activation of the NMDA ε4(NR2D) receptor; and

(c) selecting a compound for inhibiting the activation of the NMDA ε4(NR2D) receptor by comparison to the activation detected in the absence of the test compound.

8. A method of screening for a compound which binds to NMDA ε4(NR2D), the method comprising the following steps (a) to (c):

(a) contacting a test compound with a cell that expresses an NMDA ε4(NR2D) receptor gene;

(b) measuring the expression level of the NMDA ε4(NR2D) receptor gene; and

(a) selecting a compound that decreases the expression level in comparison to the level detected in the absence of the test compound.

9. A method of screening for a compound which binds to NMDA ε4(NR2D), the method comprising the following steps (a) to (c):

(a) contacting a test compound with a cell or cell extract containing DNA where the transcriptional control region of the NMDA ε4(NR2D) receptor gene is functionally linked to a reporter gene;

(b) measuring the expression level of the reporter gene; and

(c) selecting a compound that decreases the expression level of the reporter gene measured in step (b) above by comparison to the measurement conducted in the absence of a test compound.

10. A method for the determination of mechanical allodynia comprising; detecting abnormality of DNA in an NMDA ε4(NR2D) receptor gene or the control region of the gene.

11. A method for the determination of mechanical allodynia comprising a step of detecting the expression of an NMDA ε4(NR2D) receptor gene or the molecular weight of the expressed gene product.

12. A test agent for use in the determination of mechanical allodynia comprising a nucleic acid which hybridizes to an NMDA ε4(NR2D) receptor gene or the control region of the gene and contains at least the strand length of 15 nucleotides.

13. A test agent for use in the determination of mechanical allodynia comprising an antibody that binds with an NMDA ε4(NR2D) receptor protein.

14. A method of treating mechanical allodynia comprising administering a therapeutically effective amount of the pharmaceutical product according to any one of aspects 1 to 4 to a patient.

### BRIEF DESCRIPTION OF THE DRAWINGS

FIG. 1 is a graph showing the results relating to the development of mechanical allodynia (Mean±S.E.M.) with 4 NMDA ε4(NR2D) knockout mice (−/−) and 8 wild-type (WT) mice (+/+), and relates to an OPE group; and

FIG. 2 is a graph showing the results relating to the development of mechanical allodynia (Mean±S.E.M.) with 6 NMDA ε4(NR2D) knockout mice (−/−) and 8 wild-type (WT) mice (+/+), and relates to a Sham group.

### DESCRIPTION OF THE PREFERRED EMBODIMENTS

<Pharmaceutical Composition, Treatment Method>

The present invention provides a pharmaceutical composition for treating mechanical allodynia having as its active constituent a compound for inhibiting the function of an NMDA ε4(NR2D) receptor protein.

The present inventors demonstrated that inhibiting the NMDA ε4(NR2D) receptor protein is effective in relief of mechanical allodynia. Therefore, the compound for inhibiting the NMDA ε4(NR2D) receptor protein function may be used as an active constituent of the pharmaceutical composition for treating mechanical allodynia.

“Stringent hybridization conditions” is a recognized term in the art and for a given nucleic acid sequence and refers to those conditions which permit hybridization of that sequence to its complementary sequence and not to a substantially different sequence. Conditions of high stringency may be illustrated in relation to filter-bound DNA as e.g. 2×SCC, 65° C. (where SSC=0.15 M sodium chloride, 0.015 M sodium citrate, pH 7.2), or as 0.5 M NaHPO₄, 7% sodium dodecyl sulfate (SDS), 1 mM EDTA at 65° C., and washing in 0.1×SCC/0.1% SDS at 68° C. (Ausubel F. M. et al., eds, 1989, Current Protocols in Molecular Biology, Vol. I, Green Publishing Associates, Inc., and John Wiley & Sons Inc., New York, at p. 2. 10.3; incorporated herein by reference in its entirety). Hybridization conditions can be rendered highly stringent by raising the temperature and/or by the addition of increasing amounts of formamide, to destabilize the hybrid duplex of non-homologous nucleic acid sequence relative to homologous nucleic acid sequences. Thus, particular hybridisation conditions can be readily manipulated, and will generally be chosen depending on the desired results.

“Purified” does not require absolute purity; instead it is intended as a relative definition. Purification of starting materials or natural materials to at least one order of magnitude, preferably two or three orders, and more preferably four or five orders of magnitude is expressly contemplated.

“Nucleic acid or gene” means a nucleotide sequence characterized by function, any variant or homologue thereof, or truncated or extended sequence thereof, and is preferably indicated by a Genebank accession number. The terms “nucleic acid(s), nucleic acid sequence(s) or gene(s) refer interchangeably, without bias to polynucleotide sequence(s). Further, within the scope of the present invention, the term nucleic acid(s) product, or expression product or gene product or a combination of terms refers without being biased to any, protein(s), polypeptide(s), peptide(s) or fragment(s) encoded by nucleic acids, as indicated above or fragments thereof.

“Operably linked” refers to a linkage of polynucleotide elements in a functional relationship. For instance, a promoter or an enhancer is operably linked to a coding sequence if it affects the transcription of the coding sequence. In particular, two DNA molecules (such as a polynucleotide containing a promoter region and a polynucleotide encoding a desired polypeptide or polynucleotide) are said to be “operably linked” if the nature of the linkage between the two polynucleotides does not (1) result in the introduction of a frame-shift mutation and (2) interfere with the ability of the polynucleotide containing the promoter to direct the transcription of the coding polynucleotide.

The term “allodynia” as used herein is taken to mean a state where pain is felt even with stimulation that would not ordinarily cause such a pain. There are two types of allodynia; namely, chemical allodynia and mechanical allodynia. The term “mechanical allodynia” is taken to mean allodynia developed from physical action being the cause.

The term “inhibiting the NMDA ε4(NR2D) receptor protein function” as used herein is taken to mean inhibiting the intracellular signal transduction induced by the activation of the NMDA ε4(NR2D) receptor protein or in response to the activation of the NMDA ε4(NR2D) receptor protein.

The term “abnormality of DNA” as used herein refers to, for example, being due to deletion, insertion, mutation, alternative splicing, editing and so on. They have the capability to cause DNA damages.

The term “compound for inhibiting the NMDA ε4(NR2D) receptor protein function” as used herein is taken to mean (1) an antagonist of the NMDA ε4(NR2D) receptor protein, (2) a protein modulator for transducing an intracellular signal induced in response to the activation of the NMDA ε4(NR2D) receptor protein, (3) a protein or gene thereof or agonist thereof for inhibiting an intracellular signal induced in response to the activation of the NMDA ε4(NR2D) receptor protein, and (4) a molecule having a function of inhibiting the expression of an endogenous gene that codes the NMDA ε4(NR2D) receptor protein.

The terms “agonist” and “antagonist” as used herein are taken respectively to mean either a natural compound or an artificial compound with agonist or antagonist function with regard to the NMDA ε4(NR2D) receptor protein. Thus the terms “agonist” and “antagonist” includes any publicly known agonist or antagonist and also includes an agonist or antagonist isolated pursuant to a method of screening. Examples of publicly known antagonists of the NMDA ε4(NR2D) receptor protein can be selected from the group consisting of:

(±)-4-(4-phenylbenzoyl) piperazine-2,3-dicarboxylic acid (PBPD), (R,E)-4-(3-phosphonoprop-2-enyl)piperazine-2-carboxylic acid (D-CPPene), (±)-6-(1H-Tetrazol-5-ylmethyl)decahydroisoquinoline-3-carboxylic acid (LY23353), α-Amino-5-(phosphonomethyl)[1,1′-biphenyl]-3-propanoic acid (EAB515); (European Journal of Pharmacology Volume 320, Issue 1, Feb. 5, 1997, Pages 87-94); cis-4-(phosphonomethyl)piperidine-2-carboxylic acid (CGS 19755), D,L-(E)-2-amino-4-propyl-5-phosphono-3-pentenoic acid (CGP 39653), (Eur J Pharmacol Aug. 16, 1994;268(3):335-45); tanshinone IIA, tanshinone IIB; (WO 02/12218 A1), 2-(3-methylphenyl)-2-adamantanemethanamine (CEB-1604), (SFN abstracts, 2001, 27, 2, 1846); N1,N4,N8-tri-benzyl-spermidine (TB-34), (J Pharmacol Exp Ther 1997 283: 533-540); and Memantine, (SFN abstracts, 2001, 27, 2, 1846); among others. By way of further example an “agonist”or “antagonist” can include an antibody that binds to the NMDA ε4(NR2D) receptor protein.

In relation to molecules having a function of inhibiting the expression of an endogenous gene that codes the NMDA ε4(NR2D) receptor protein the term “compound for inhibiting the NMDA ε4(NR2D) receptor protein function” as used herein is taken to refer to the following: antisense DNA (DNA that codes antisense RNA complementary to a transcriptional product of a gene that codes the NMDAε4(NR2D) receptor protein), DNA that codes ribozyme (RNA having ribozyme activity for specifically cleaving the transcriptional product of a gene that codes the NMDA ε4(NR2D) receptor protein).

Preferably the antisense DNA sequence is a sequence complementary to the NMDA ε4(NR2D) receptor gene or a part thereof, so as long as the gene expression can be effectively inhibited although it does not have to be completely complementary. Preferably the transcribed RNA has a complementarity of 90% or more, more preferably a complementarity of 95% or more to the transcriptional product of the target gene. Preferably the antisense sequence has at least a strand length of 15 bp, more preferably 100 bp, increasingly preferably 500 bp or more. Furthermore the antisense sequence preferably has a strand length of 3000 bp or less, more preferably 2000 bp or less. The antisense DNA, for example, may be prepared with the likes of a phosphorothionate method (Stein, 1988 Physicochemical properties of phosphorothioate oligodeoxynucleotides. Nucleic Acids Res 16, 3209-21 (1988)) based on the sequence information of the DNA that codes the NMDA ε4(NR2D) receptor protein.

A ribozyme is an RNA molecule having catalytic activity. Ribozymes have various activities, and the engineering of ribozymes directed at the site-specific cutting of RNA has become possible pursuant to the research of ribozymes as an enzyme for cutting RNA. Among the ribozymes, there are those such as the group I intron type and MIRNA contained in RnaseP having 400 nucleotides or more, and there are those referred to as a hammerhead type or hairpin type having an activated domain of approximately 40 nucleotides (Makoto Koizumi and Eiko Otsuka, (1990) Protein, Nucleic Acid and Enzyme, 35: 2191).

For example, although the autonomy cutting domain of a hammerhead ribozyme cuts the 3' side of C15 of G13U14C15, it is essential that U14 forms a base pair with the ninth A, and it has reported that the fifteenth base is also capable of being cut with A or U in addition to C (M. Koizumi et al., (1988) FEBS Lett. 228: 225). If the substrate-binding site of the ribozyme is engineered to be complementary to the RNA sequence in the vicinity of the target site, it is possible to create a restricted enzyme RNA cutting ribozyme capable of recognizing the sequence of UC, UU or UA among the target RNA (M. Koizumi et al., (1988) FEBS Lett. 239: 285, Makoto Koizumi and Eiko Otsuka, (1990) Protein, Nucleic Acid and Enzyme, 35: 2191, M. Koizumi et al., (1989) Nucleic Acids Res. 17: 7059). There are plural sites that could be a target of such RNA cutting ribozyme in the RNA sequence that codes the NMDA ε4(NR2D) receptor protein.

Further, hairpin ribozymes are also useful for the objective of the present invention. Hairpin ribozymes, for instance, are discovered in the minus strand of a satellite RNA of a tobacco ring spot virus (J. M. Buzayan Nature 323: 349, 1986). It has also been reported that this ribozyme can be engineered so as to yield target-specific RNA cutting (Y. Kikuchi and N. Sasaki (1992) Nucleic Acids Res. 19: 6751, Hiroshi Kikuchi, (1992) Bioscience and Biotechnology 30: 112).

In relation to molecules having a function of inhibiting the expression of an endogenous gene that codes the NMDA ε4(NR2D) receptor protein the term “compound for inhibiting the NMDA ε4(NR2D) receptor protein function” as used herein is taken to refer to the foregoing antisense DNA and DNA that codes such ribozymes which may be used in the gene therapy of mechanical allodynia.

The “compound for inhibiting the NMDA ε4(NR2D) receptor protein function” as referred to in aspects 1 to 4 can be administered alone but, in human therapy, will generally be administered in admixture with a suitable pharmaceutical excipient or carrier selected with regard to the intended route of administration and standard pharmaceutical practice as a pharmaceutical composition. A.pharmaceutical composition of the present invention may be administered to a patient, in combination with a carrier, once or a plurality of times. An appropriate medical carrier includes an inactive solid diluent or extender, an aseptic aqueous solution and various organic solvents. The pharmaceutical composition may be administered in various dosage forms such as a tablet, powder, lozenge, syrup, injectable solution, and other similar items. If so desired, an additional component such as a flavoring agent, a binder, an adjuvant or a similitude thereof may be added to these pharmaceutical compositions. As described above, in consideration of oral administration, tablets containing various adjuvants such as sodium citrate, calcium carbonate and calcium phosphate may be employed together with binders such as polyvinyl pyrrolidone, sucrose, gelatin and acacia upon being added thereto various disintegrators such as starch, methyl cellulose, alginic acid and compound silicate. Further, lubricants such as magnesium stearate, sodium lauryl sulfate and talc are at times useful for the manufacture of tablets. Moreover, an isomorphic solid composition may be employed as an extender for filling a soft- and hard-gelatin capsule. A substance desirable therefor includes lactose or milk sugar, and high molecular weight polyethylene glycol. When an aqueous suspension or elixir is desired for the oral administration, the essential active ingredients thereof may be combined with a sweetening agent or flavoring agent, a coloring substance or dye, and, if so desired, with an emulsifying agent or suspension, together with a diluent such as water, ethanol, propylene glycol, glycerin and the combinations thereof.

With parenteral (non-oral) administration, a solution containing salt capable of incorporating an active compound of the present invention or as a pharmaceutical thereof may be employed in sesame oil or peanut oil, aqueous propylene glycol, or aseptic aqueous solution. This type of aqueous solution must be appropriately buffered as necessary, and the liquid diluent was initially made isotonic with sufficient salt solution or glucose. Such specific aqueous solutions are in particular adequate for intravenous administration, intramuscular administration, subcutaneous administration, and intraperitoneal administration. The aseptic aqueous solution to be employed may be easily obtained with standard technology publicly known to those skilled in the art.

With regard to gene therapy, when administering a therapeutic gene in vivo, a virus vector such as a retrovirus, adenovirus or Sendai virus or a non-virus vector such as liposome may be used. An in vivo method and ex vivo method may be exemplified as methods of administration.

Pharmaceutical compositions are to be administered to a patient in a dosage effective for the treatment of mechanical allodynia. The dose may vary depending on various factors such as the patient's age, weight, symptom, method of administration, and so on. An experienced physician may appropriately select the adequate dose.

<Screening>

The present invention provides a screening method for a pharmaceutical candidate compound for treating mechanical allodynia.

The screening method may comprise the detection of a bonding interaction between the NMDA ε4(NR2D) receptor protein and a candidate or test compound when the NMDA ε4(NR2D) receptor protein and test compound are brought into contact. The NMDA ε4(NR2D) receptor protein may be in the form in which it is expressed intracellularly, alternatively it may be expressed on the cell surface or in a form contained in the cell membrane faction of such cell. Preferably the NMDA ε4(NR2D) receptor protein is in a form of bound to an affinity column, most preferably the NMDA ε4(NR2D) receptor protein is in purified form. Additionally the test compound to be employed in this method may be suitably labeled as necessary upon usage.

An example of the screening method may include the detection of the interaction between the NMDA ε4(NR2D) receptor protein and test compound using a label affixed to the test compound.

In the present method, the test compound binding with the NMDA ε4(NR2D) receptor protein is selected. In an example of the present screening method, an agonist and antagonist may be included in the compound isolated as the compound to be bonded with the NMDA ε4(NR2D) receptor protein.

In a further example of the screening method, a test compound may be brought into contact with the NMDA ε4(NR2D) receptor protein which is expressed on a cell surface in order to determine whether an intracellular signal transduction, which is to be the indicator of the protein activation, occurs, this method permits the evaluation of whether a certain test compound is an agonist of the NMDA ε4(NR2D) receptor protein. Preferably the ε4(NR2D) protein and ζ protein in combination form the ion channel on the cell membrane, and, by the agonist being bonded thereto, the channel opens allowing extracellular ion exchange to occur. There are preferably three types of ions that permeate this channel; namely, Na⁺, Ca²^{₊} and K⁺, but the permeation of Ca²^{₊} is in particular characteristic. Therefore, the inflow of Ca²^{₊} into the cells is the most preferable indicator of activation of the NMDA ε4(NR2D) receptor protein. The inflow of Ca²^{₊} into the cells is preferably detected by the measurement of the fluorescent variation with a calcium-selective fluorescent chelator.

In an alternative example of the screening method, and without limitation to Ca²^{₊}, the sum of all cation transportsation may be employed as the indicator of the activation of the NMDA ε4(NR2D) receptor protein. The sum of cation transportation may be preferably detected through measurement of the cell membrane permeation current or measurement of the membrane potential variation with the electrophysiologic method. In the abovestated example of the screening method a compound that generates this kind of intracellular signal transduction may be considered to be an agonist of the NMDA ε4(NR2D) receptor protein.

By way of a further example of the screening method it is possible to evaluate whether the isolated test compound is an antagonist, by bringing a test compound into contact, in the presence of a ligand, with the NMDA ε4(NR2D) receptor protein expressed on the cell surface and by using the intracellular signal transduction as an indicator of activation of the receptor protein. In this example a test compound that inhibits the intracellular signal transduction response due to the ligand stimulation may be considered to be an antagonist of the NMDA ε4(NR2D) receptor protein. A manner of screening an agonist or antagonist of the NMDA ε4(NR2D) receptor protein by utilizing the intracellular signal pathway occurring pursuant to the binding of the test compound to the cell surface expressed NMDA ε4(NR2D) receptor protein is therefore also an example of the screening method of the present invention. The antagonist identified by the above mentioned manner will be a candidate of the drug for treating mechanical allodynia.

A further example of the screening method according to the present invention is a method of makes use of the expression of the NMDA ε4(NR2D) receptor gene the indicator of interaction of a test compound with the receptor. The present inventors have demonstrated that the inhibition of the NMDA ε4(NR2D) receptor gene expression relates to the improvement of mechanical allodynia. Therefore, a compound that reduces the expression of a normal NMDA ε4(NR2D) receptor gene is anticipated to become a pharmaceutical candidate for treating mechanical allodynia. In this example of the present method, a test compound is preferably brought into contact with a cell that expresses the NMDA ε4(NR2D) receptor gene. The origin of the employed “cell” may be a cell originating from a human, monkey, mouse, rat, cattle, swine, dog, among others, but is not limited to the foregoing.

There is no particular limitation on the test compound to be used in the present method. For example, a natural compound, organic compound, inorganic compound, protein, single compound such as peptide, as well as a compound library, expression product of a gene library, cell extract, cell culture supernatant, fermented microorganism product, marine organism extract, vegetable organism extract and so on may be exemplified, but the test compound is not limited thereto.

The term “contact” as used herein is taken to the process of adding a test compound to the culture solution of the cell that expresses the NMDA ε4(NR2D) receptor gene, but is not limited thereto. When the test compound is protein or the like, the term “contact” may be understood to mean the process of introducing the DNA vector that expresses such protein into the cell.

The term “gene expression” as used herein is taken to refer to both both transcription and translation.

In a further example of the screening method, the expression level of the NMDA ε4(NR2D) receptor gene is measured. Measurement of the gene expression level may be conducted with a method publicly known to those skilled in the art. For example, the transcription level of the gene may be measured by extracting mRNA in accordance with a standard method from the cell that expresses the NMDA ε4(NR2D) receptor gene and performing the northern hybridization method or RT-PCR method with this mRNA as the genetic template. Alternatively, it is also possible to measure the translation level of the gene by collecting the protein fraction from the cell that expresses the NMDA ε4(NR2D) receptor gene and detecting the expression of the NMDA ε4(NR2D) receptor protein with electrophoresis such as SDS-PAGE. In a further example of the present screening method possible the translation level of the gene may be measured by detecting the expression of the expressed protein upon performing the western blotting method with an antibody against the NMDA ε4(NR2D) receptor protein. There is no particular limitation on the antibody to be employed in the detection of the NMDA ε4(NR2D) receptor protein so as long as it is a labeled detectable antibody, and this may be a monoclonal antibody or a polyclonal antibody.

In a further example of the present screening method a compound may be selected that reduces the expression level NMDA ε4(NR2D) receptor gene in comparison to a case of not bringing the test compound into contact (control). The compound selected as described above will become a pharmaceutical candidate for treating mechanical allodynia.

Another example of the screening method according to the present invention relates to a method of identifying a compound capable of decreasing the expression of the NMDA ε4(NR2D) receptor gene as detected by utilizing a reporter gene. In this example of the present method a test compound is brought into contact with a cell or cell extract containing DNA wherein the transcription control region of the NMDA ε4(NR2D) receptor gene and the reporter gene are functionally bonded.

Here, “functionally bonded” means that the transcriptional control region of the NMDA ε4(NR2D) receptor gene and the reporter are linked so that the expression of the reporter gene may be induced by a transcription factor interacting with the transcriptional control region of the NMDA ε4(NR2D) receptor gene. The transcriptional control region of the NMDA ε4(NR2D) receptor gene could be obtained by the person who is skilled in the art, by screening the genome DNA library by using probes designed from a part or the whole of the NMDA ε4(NR2D) receptor gene, or conducting polymerase chain reaction using the genome DNA as template and primers designed based on a sequence of the transcription control region of the NMDA ε4(NR2D) receptor gene.

There is no particular limitation on the reporter gene employed in the present screening method so as long as the expression can be detected. Examples of suitable reporter systems include: a CAT gene, lacZ gene, luciferase gene, GFP gene, among others.

Examples of a “cell containing DNA having a construct in which the transcription control region of the NMDA ε4(NR2D) receptor gene and the reporter gene are functionally bonded” include any cell in which a vector having the foregoing construct inserted therein is introduced into such cell. This kind of vector may be prepared by standard methods publicly known to those skilled in the art. Introduction of the vector into the cell may be performed with a standard method, for instance, the calcium phosphate precipitation method, electrical pulse terebration, lipophetamine method, microinjection method, among other methods. A “cell containing DNA having a structure in which the transcription control region of the NMDA ε4(NR2D) receptor gene and the reporter gene are functionally bonded” includes any cell in which the foregoing structure is inserted into a chromosome. Insertion of the DNA structure into the chromosome may be conducted using standard methods generally employed by a person who is skilled in the art; for example, the congenic method utilizing homologous splicing.

* The term “contact” as used herein with reference to reporter gene constructs is taken to mean adding a test compound to the culture solution of a “cell containing DNA containing a construct in which the transcription control region of the NMDA ε4(NR2D) receptor gene and the reporter gene are functionally bonded”, or alternatively adding a test compound to a cell extract containing such a DNA construct. The term “contact” as used herein with reference to reporter gene constructs and in the case when the test compound is a protein, is taken to mean inserting a vector that expresses such protein into the cell.

The reporter gene expression level may be measured by a method publicly known to a person who is skilled in the art in accordance with the type of reporter gene. For example, when the reporter gene is a CAT gene, the reporter gene representation may be measured by detecting the acetylation of chloramphenicol pursuant to the gene product. When the reporter gene is a lacZ gene, the reporter gene representation may be measured by detecting the coloring of the pigment compound caused by the catalysis of the gene expression product, when the reporter gene is a luciferase gene, the reporter gene representation may be measured by detecting the fluorescence of the fluorescent compound caused by the catalysis of the gene expression product, and, when the reporter gene is a GFP gene, the reporter gene representation may be measured by detecting the fluorescence caused by the GFP protein.

In present example of the present method a compound may be selected for decreasing the measured reporter gene expression level when compared with a case of measuring reporter gene expression levels in the absenceof the test compound. The compound selected as described above will become a pharmaceutical candidate for treating mechanical allodynia.

*<Inspection Method/Test Agent>

The present invention provides an method for the determination of mechanical allodynia. In the present embodiment, a mouse with the NMDAε4(NR2D) receptor gene knocked out showed an improved symptom of mechanical allodynia. This fact shows that the mutation or expression of the NMDA ε4(NR2D) receptor gene is involved in the development of the symptoms of mechanical allodynia. Therefore, mechanical allodynia may be determined by analyzing the mutation or expression of the NMDA ε4(NR2D) receptor gene.

The term “determination of mechanical allodynia” as used herein refers to the inspection of a subject showing a symptom of mechanical allodynia and determining it as originating from the mutation or expression abnormality of the NMDA ε4(NR2D) receptor gene, and using this determination as an indication for judging whether the subject is susceptible to the development of mechanical allodynia.

An example of a method for the determination of mechanical allodynia includes determining the base sequence of the subject's NMDA ε4(NR2D) receptor gene preferably by preparing a DNA sample from the subject in order to detect mutation or expression abnormality of the NMDA ε4(NR2D) receptor gene. The DNA sample may be prepared, for example, from the chromosomal DNA or RNA as extracted from the tissue or cell of the subject. Preparation of a DNA sample may be achieved from chromosomal DNA and a genome library may be prepared by cutting the chromosome DNA with an appropriate restriction enzyme and cloning this resticted DNA into a vector. In order to prepare a DNA sample of the present method from RNA, for example, a cDNA library may be prepared from RNA with reverse transcriptase according to standard methods as commonly known to those skilled in the art. Subsequently, DNA that codes the NMDAε4(NR2D) receptor gene originating from the subject is selected.

The term “select” as used herein as used in reference to DNA isolation is taken to mean to specifically isolate the DNA that codes the NMDA ε4(NR2D) receptor gene (a part or the whole of the NMDA ε4(NR2D) receptor gene or the gene control region of the subject). The selection of DNA that codes the NMDA ε4(NR2D) receptor gene may be conducted by screening the genome library or cDNA library employing a probe to be hybridized to the DNA that codes the NMDA ε4(NR2D) receptor gene. It is preferable to select the DNA that codes the NMDA ε4(NR2D) receptor gene from a genome DNA library or from a cDNA library or by using PCR having RNA as the genetic template and using a primer to be hybridized to the DNA that codes the NMDA ε4(NR2D) receptor gene.

* In one example of the method of determination of mechanical allodynia, the selected DNA base sequence is determined. Determination of the selected DNA base sequence may be conducted with a method publicly known to those skilled in the art. The determined DNA base sequence is then compared with the control which is the sequence of a normal (wild type) NMDA ε4(NR2D) receptor gene or the gene control region. Since a healthy individual's sequence of NMDA ε4(NR2D) receptor gene or the gene control region of said gene should be considered to be normal, preferably the aforementioned step of comparison with the control ordinary means the comparison with the sequence of the NMDA ε4(NR2D) receptor gene or the gene control region of a generally healthy individual. Alternatively, the comparison of the sequence of the NMDA ε4(NR2D) receptor gene may also be made with a sequence of the NMDA ε4(NR2D) receptor gene or the gene control region that is registered as a wild type in GenBank or the like. If the NMDA ε4(NR2D) receptor gene or the gene control region of the subject differs from the control as a result of the foregoing comparison, such subject is judged to be suspected of having mechanical allodynia based on the mutation of the NMDA ε4(NR2D) receptor gene or the gene control region.

A further example of the method for the determination of mechanical allodynia may employ alternative methods of determining the DNA base sequence of the subject the NMDA ε4(NR2D) receptor gene sequence. For example the subject DNA sample, preferably the DNA that codes the NMDA ε4(NR2D) receptor gene originating from the subject, may be cut with a restriction enzyme. Next, the resulting DNA fragments may be separated in accordance with their size. Then, the size of the detected DNA fragments is compared with the control in order to determine whether there is any abnormality in the subject DNA. Alternatively a DNA sample may be prepared from the subject and the DNA that codes the NMDA ε4(NR2D) receptor gene originating from the subject is amplified with the DNA as a primer. Further, the amplified DNA may be cut with a restriction enzyme and the DNA fragments separated in accordance with their size. Then, the size of the detected DNA fragments may be compared with the control in order to determine whether there is any abnormality in the subject DNA.

A further example of the method for the determination of mechanical allodynia may employ Restriction Fragment Length Polymorphism (RFLP) or the PCR-RFLP method to detect mutation. For example if a mutation exists in the recognition site of the restriction enzyme, or when there is a base insertion or deficiency within the DNA fragment arising from the restriction enzyme treatment, the size of the fragment arising after the restriction enzyme processing changes in comparison to the control. Further to this the site containing the mutation can be amplified with the PCR method, and, by performing processing with the respective restriction enzymes, these mutations may be detected as the difference in the mobility of the band after electrophoresis between subject and control. Alternatively, the chromosomal DNA may be processed with these restriction enzymes, and, after electrophoresis, the existence of mutation may be detected by performing southern blocking with the probe DNA of the present invention. The restriction enzyme used may be suitably selected in accordance with each mutation. By using this method, it is possible to do southern blotting on cDNA, other than genome DNA, after cutting such cDNA with a restriction enzyme, for which such cDNA is obtained from RNA by reverse transcription that is prepared from the subject. Further, it is also possible to amplify a part or the whole of the NMDA ε4(NR2D) receptor gene with PCR in which this cDNA is the genetic template, cut this with a restriction enzyme, and thereafter inspect the difference in mobility by gel electrophoresis.

In a further example of the method for the determination of mechanical allodynia, a DNA sample is prepared from the subject. Subsequently, the DNA that codes the NMDA ε4(NR2D) receptor gene originating from the subject is amplified with the DNA as a primer. The amplified DNA is disassociated as a single strand DNA. Next, the disassociated single strand DNA is separated on the undenatured gel. The mobility on the gel of the separated single strand DNA is compared with the control.

A further example of the method for the determination of mechanical allodynia may employ the PCR-SSCP (single-strand conformation polymorphism) method (Cloning and polymerase strand reaction-single-strand conformation polymorphism analysis of anonymous Alu repeats on chromosome 11. Genomics. Jan. 1, 1992; 12(1): 139-146, Detection of p53 gene mutations in human brain tumors by single-strand conformation polymorphism analysis of polymerase strand reaction products. Oncogene. Aug. 1, 1991; 6(8): 1313-1318, Multiple fluorescence-based PCR-SSCP analysis with postlabeling., PCR Methods Appl. Apr. 1, 1995; 4(5): 275-282.). The operation of this PCR-SSCP method is relatively easy, and, since there is an advantage in that the amount of the subject sample required is minimal, this is particularly preferable when screening numerous DNA samples. The principle is as follows. When a double stranded DNA fragment is disassociated into a single strand, each strand forms a unique higher order structure dependent on the base sequence thereof. When this disassociated DNA strand is subject to electrophoresis in a polyacrylamide gel that does not contain a denaturant, a single strand DNA having the same complementary strand length moves to a different position in accordance with the difference in the respective higher order structures. The higher order structure of this single strand DNA changes even with a monobasic substitution, and shows a different mobility in the polyacrylamide gel electrophoresis.

In a further example of the method for the determination of mechanical allodynia a part or the whole of the NMDA ε4(NR2D) receptor gene is amplified with the PCR method or the like. It is preferable that the amplified range is normally between a length of approximately 200 to 400 bp. Moreover, included in the amplified region are the entire exon and entire intron of the NMDA ε4(NR2D) receptor gene, as well as the promoter and enhancer of the NMDA ε4(NR2D) receptor gene. PCR may be performed by a person who is skilled in the art upon suitably selecting the reactive condition. Upon PCR, an isotope of ³²P or the like, fluorochrome, or primer labeled with a marker such as of biotin may be used in order to label the amplified DNA product. Or, by performing PCR upon adding an isotope of ³²P or the like, fluorochrome, or substrate base labeled with the likes of biotin to a PCR reaction liquid, it is also possible to label the amplified DNA product. Additionally, by adding an isotope of ³²P or the like, fluorochrome, or substrate base labeled with, for example, biotin to the amplified DNA fragment with a Klenow fragment or the like after the PCR reaction, it is also possible to label the amplified DNA product. The indicated DNA fragment obtained above is heat dissociated, and electrophoresis is performed with polyacrylamide gel that does not contain a denaturant such as urea. Conditions for separating the DNA fragment may be improved by adding an adequate dose (roughly 5 to 10%) of glycerol to the polyacrylamide gel. Moreover, although the electrophoresis conditions will vary depending on the property of the respective DNA fragments, electrophoresis is usually conducted at room temperature (between 20 to 25° C.), and, when favorable separation cannot be achieved, the temperature is set between 4 to 30° C. in order to provide optimum mobility. After electrophoresis, mobility of the DNA fragment is detected and analyzed with the likes of autoradiography employing an X-ray film or a scanner for detecting fluorescence. When a band having a difference in the mobility with respect the control is detected, this band is directly removed from the gel, re-amplified with PCR, and, through direct sequencing, the existence of mutation may be confirmed with respect to the control sequence. Further, even in a case of not using the labeled DNA, the band may be detected by dyeing the gel after electrophoresis with the likes of ethidium bromide or the silver impregnation method.

In a further example of the method for the determination of mechanical allodynia a DNA sample is prepared from the subject. Subsequently, the DNA that codes the NMDA ε4(NR2D) receptor gene originating from the subject is amplified with the DNA as a primer. Next, the amplified DNA is separated on a gel in which the concentration of the DNA denaturant gradually increases. Then, the mobility on the gel of the separated DNA is compared with the control. This protocol is more specifically exemplified by the denaturant gradient gel electrophoresis (DGGE) method. The DGGE method is a method of electrophoresing the DNA fragment mixture within the polyacrylamide gel having a denaturant gradient, and separating the DNA fragment as a result of the difference in each fragment's instability. When an instable DNA fragment having a mismatch moves to a site of a certain denaturant concentration within the gel, the DNA sequence in the periphery of the mismatch will partially disassociate to a single strand. The mobility of this partially disassociated DNA fragment will become extremely low, and, since there will be a difference in the mobility in comparison to a complete double strand DNA without any disassociated site, the two may be separated. For example, a part or the whole of the NMDA ε4(NR2D) receptor gene is amplified with PCR method or the like by employing the primer or the like prepared in the present invention, then the results of such amplification are compared with the control by electrophoresis within the polyacrylamide gel in which the concentration of a denaturant such as urea is gradually increasing in accordance with the migration of the fragments. In the case of a DNA fragment where mutation exists, the DNA fragment becomes a single strand at a lower denaturant concentration position, and, since the mobility speed becomes extremely slow, the existence of mutation can be detected by detecting the difference in this mobility.

A further example of the method for the determination of mechanical allodynia may employ a mass spectrograph (MASS). Firstly, a DNA sample is prepared from the subject. Subsequently, the DNA that codes the NMDA ε4(NR2D) receptor gene originating from the subject is amplified. Further, the amplified DNA is separated with a mass spectrograph. Next, the mass of the separated subject DNA is compared with the control.

In addition to the foregoing examples, the Allele Specific Oligonucleotide (ASO) hybridization method may be used for the purpose of detecting mutation of a specific position in subject DNA. In the method an oligonucleotide containing a base sequence in which mutation is known to exist is hybridized with the sample subject DNA, if mutation exists in the subject DNA the hybridization efficiency will decrease. This hybridization may be detected with the southern blotting method, or a method utilizing the property in which a special fluorescence reagent is optically quenched through intercalation in the hybrid gap. Moreover, detection is also possible with the ribonuclease A mismatch cutting method. Specifically, a part or the whole of the NMDA ε4(NR2D) receptor gene is amplified with the likes of a PCR method, and this is hybridized with the labeled RNA prepared from the likes of cDNA that codes the NMDA ε4(NR2D) receptor protein implanted in a plasmid vector or the like. Since the hybrid will be of a single strand structure in the site where mutation exists, this site is easily cut with ribonuclease A, and the existence of mutation can be detected by detecting this with autoradiography or the like.

A further example of the method for the determination of mechanical allodynia comprises a method of using the expression of the gene that codes the NMDA ε4(NR2D) receptor or the molecular weight of the expression product as an indicator of mechanical allodynia.

The term “gene expression” as used herein includes both transcription and translation, therefore, MRNA and protein are included in the “expression product”.

In order to determine the transcription level of the gene that codes the NMDA ε4(NR2D) receptor protein an RNA sample is prepared from the subject. Subsequently, the quantity or molecular weight of the RNA that codes the NMDA ε4(NR2D) receptor protein contained in the RNA sample is detected. Next, the quantity or molecular weight of the detected RNA is compared with the control. RNA detection can be achieved by the northern blotting method employing a probe which can hybridize with the DNA that codes the NMDA ε4(NR2D) receptor protein, RT-PCR and employing a primer hybridizable with the DNA that codes the NMDA ε4(NR2D) receptor protein, and DNA microarray method hybridizable with oligonucleotide.

The translation level of the gene that codes the NMDA ε4(NR2D) receptor protein can be determined by the following methods a protein sample is prepared from the subject, subsequently, the quantity or molecular weight of the NMDA ε4(NR2D) receptor protein contained in the protein sample is detected. Next, the quantity or molecular weight of the detected protein is compared with the control. An exmple of this type of methodology is the SDS polyacrylamide electrophoresis method, as well as the western blotting method, dot blotting method, immunoprecipitation method, Enzyme Linked ImmunoSorbent

Assay (ELISA), and immunnofluorescence employing an antibody binding with the NMDA ε4(NR2D) receptor protein.

By application of the above examples of the method for the determination of mechanical allodynia, when the expression of the gene that codes the NMDA ε4(NR2D) receptor protein is significantly rising or falling in comparison to the control (healthy individual), or if the molecular weight of the expression product is significantly different, the subject is judged to have mechanical allodynia.

The present invention additionally provides a test agent to be employed in the determination of mechanical allodynia.

An example of a test agent is oligonucleotide having at least a strand length of 15 nucleotides and capable of hybridizing to the NMDA ε4(NR2D) receptor gene or the control region of such gene.

Preferably, this oligonucleotide is specifically hybridizable to the base sequence of the NMDA ε4(NR2D) receptor gene or the control region of such gene.

The term “specifically hybridize” as used herein is taken to mean cross hybridization which does not occur significantly with the DNA that codes for other proteins under a normal hybridization conditions, preferably under a stringent hybridization condition (for example, the condition described in 2^{nd} Edition, 1989, Sam Brook et al., Molecular Cloning, Cold Spring Harbour Laboratory Press, New York, USA).

A hybridizing oligonucleotide may be employed as the probe or primer in the method of the present invention described above. When employing this oligonucleotide as a primer, the length thereof is usually 15 bp to 100 bp, and preferably 17 bp to 30 bp. There is no particular limitation on the primer so as long as it is capable of hybridizing to at least a part of the NMDA ε4(NR2D) receptor gene or the gene control region. An example of the may be the exon region, intron region, promoter region, enhancer region and so on of the NMDA ε4(NR2D) receptor gene.

When using an oligonucleotide as a probe, there is no particular limitation on the probe so as long as it specifically hybridizes to at least a part of the NMDA ε4(NR2D) receptor gene or the gene control region. The probe may be a synthetic oligonucleotide and normally has a strand length of at least 15 bp or more. An example of the target hybridization region may be the exon region, intron region, promoter region, enhancer region and so on of the NMDA ε4(NR2D) receptor gene.

The oligonucleotide of the present invention may be prepared, for example, with a commercially available oligonucleotide synthesizer. The probe may also be prepared as a double strand DNA fragment acquired with restricted enzyme processing or the like.

When using the oligonucleotide of the present invention as a probe, it is preferable that it is labeled adequately. An example of a method of labeling is the labeling method of phosphorylating the oligonucleotide 5' end with ³²P with a T4 polynucleotidekinase, and a method of incorporating the substrate base indicated pursuant to an isotope such as ³²P, fluorochrome, or biotin with a random hexamer oligonucleotide or the like as the primer using a DNA polymerase such as a Klenow fragment (the so called random prime method).

A further example of the test agent according to the present invention is a test agent comprising an antibody that binds to the NMDA ε4(NR2D) receptor protein. There is no particular limitation on the antibody used for example it may be may be a polyclonal antibody or a monoclonal antibody. The antibody may be labeled as necessary.

An antibody that binds to the NMDA ε4(NR2D) receptor protein may be prepared using a method publicly as known to those skilled in the art. In the case of a polyclonal antibody it may be obtained as follows; a recombinant NMDA ε4(NR2D) receptor protein or its partial peptide may be expressed in a microorganisim such as Escherichia coli as the fusion protein with GST. This is used to immunized a small animal such as a rabbit or the like from which blood serum is subsequently obtained. Serum is separated from the whole blood and the antibody purified by for example affinity purification or ammonium sulfate precipitation, or purification on a protein A or protein G column, DEAE ion exchange chromatography, or a column bearing NMDA ε4(NR2D) receptor protein as a synthetic peptide. If a monoclonal antibody, is intended, the NMDA ε4(NR2D) receptor protein or its partial peptide is immunized in a small animal such as a mouse, the spleen is removed from such mouse, and spleen cells obtained from the spleen by homogenization of said spleen. These cells and the mouse myeloma cells are fused with a reagent such as polyethylene glycol, and a clone for producing the antibody that binds to the NMDA ε4(NR2D) receptor protein is selected among the hybridoma formed thereby. Subsequently, the obtained hybridoma is transferred into the abdominal cavity, hydroperitoneum is collected from the mouse, and the obtained monoclonal antibody is prepared through purification by an affinity column or the like coupling ammonium sulfate precipitation, protein A, protein G column, DEAE ion exchange chromatography, NMDA ε4(NR2D) receptor protein and synthetic peptide.

A test agent of the present invention, for example oligonucleotide or antibody, may be used in combination with, sterilized water, isotonic sodium chloride solution, vegetable oil, surface active agent, lipid, solubilizer, buffer, protein stabilizer (BSA, gelatin, and so on), preservative and so on may be mixed therein as necessary.

### EXAMPLES

The present invention is explained in further detail with reference to the Examples. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### Example 1

### Knockout Effect of NMDA ε4(NR2D) Receptor Gene in Seltzer Model

The test employed a C57BL/6 mouse (hereinafter referred to as a “knockout mouse”) having a deficient NMDA ε4(NR2D) receptor gene. A Seltzer model mouse was prepared from the foregoing mouse as follows. The sciatic nerve was exposed at the crural area under isoflurane anesthesia, approximately ½ of the sciatic nerve was tied up with a 9-0 silk thread, and the wound was closed layer to layer. Meanwhile, a Sham group in which only the sciatic nerve is not tied up was simultaneously prepared.

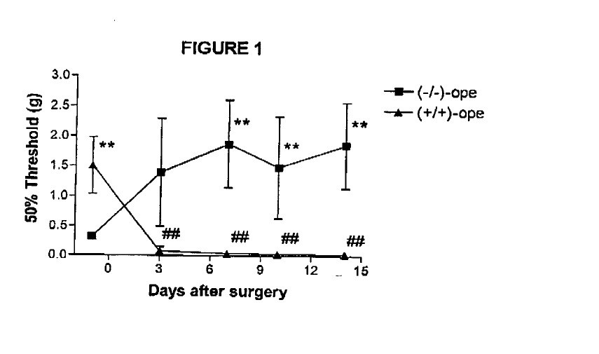

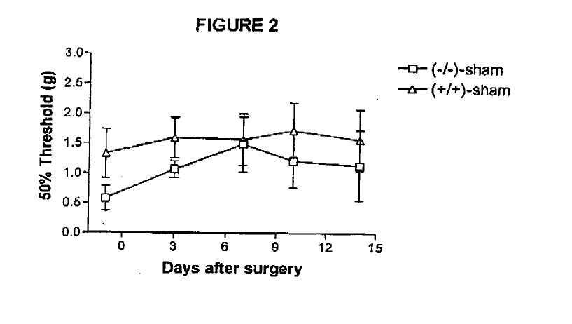

The allodynia activity of the mouse prior to the operation and two weeks after the operation was evaluated with the up/down method employing the “von Frey hair test (a test conducted by applying a filament from underneath perpendicularly to the plantar of the hind paw and examining the withdrawal response of the paw)”. In order to inspect the allodynia activity on a day-by-day basis, a total of 5 von Frey hair tests; namely, pre, third day, seventh day, tenth day, fourteenth day, were conducted for each mouse in this experiment. And, based on this experiment, a 50% response threshold (von Frey hair threshold) was calculated upon using the formula reported by Chaplan et al. (Chaplan S R, Bach F W, Pogrel J W, Chung J M, Yaksh T L) Quantitative assessment of tactil allodynia in the rat paw, (J. Neurosci. Methods 53: 55-63, 1994). The results relating to the development of mechanical allodynia (Mean±S.E.M.) with knockout mice (−/−) and wild type (WT) mice (+/+) are shown in FIG. 1 and FIG. 2. Further, in FIG. 1 and FIG. 2, the vertical axis shows the von Frey hair threshold. This implies that the smaller the threshold, the more serious the degree of development of mechanical allodynia. Moreover, FIG. 1 is data of the mouse group (OPE group) in which the sciatic nerve has been tied up, and FIG. 2 is data of the mouse group (Sham group) in which the sciatic nerve has not been tied up.

With the OPE group, the WT mouse, in comparison to pre, showed a significant decrease in the von Frey hair threshold in days 3, 7, 10 and 14 after the operation. With the knockout mouse, in comparison to pre, no significant change in the threshold could be detected. When comparing the thresholds of the knockout mouse and the WT mouse in the respective test dates, a significant difference is evident in pre, day 7, day 10 and day 14. In other words, it is clear that the knockout mouse has a higher von Frey hair threshold in comparison to the WT mouse in all test days after the operation. Accordingly, it has been demonstrated that mechanical allodynia does not develop in a mouse deficient in the NMDA ε4(NR2D) gene.

In addition, it is clear that with the Sham group, no significant difference could be recognized in the comparison of the von Frey hair threshold in pre and the respective treatment days.

1. A method of treating mechanical allodynia comprising administering to a therapeutically effective amount of a compound which inhibits the function of an NMDA ε4(NR2D) receptor protein.

2. A method according to claim 1, wherein the compound for inhibiting the function of an NMDA ε4(NR2D) receptor protein is an antagonist of the NMDA ε4(NR2D) receptor protein.

3. A method according to according to claim 2, wherein the antagonist is selected from the group consisting of
(±)-4-(4-phenylbenzoyl) piperazine-2,3-dicarboxylic acid (PBPD); (R,E)-4-(3-phosphonoprop-2-enyl)piperazine-2-carboxylic acid (D-CPPene); (±)-6-(1H-Tetrazol-5-ylmethyl)decahydroisoquinoline-3-carboxylic acid (LY23353); α-Amino-5-(phosphonomethyl)[1,1′-biphenyl]-3-propanoic acid (EAB515); cis-4-(phosphonomethyl)piperidine-2-carboxylic acid (CGS 19755); D,L-(E)-2-amino-4-propyl-5-phosphono-3-pentenoic acid (CGP 39653); Tanshinone IIA; tanshinone IIB; 2-(3-methylphenyl)-2-adamantanemethanamine (CEB-1604); N1,N4,N8-tri-benzyl-spermidine (TB-3-4); and Memantine.

4. A pharmaceutical composition for treating mechanical allodynia comprising a compound as defined in claim 1 and a pharmaceutically acceptable diluent or carrier.

5. A method of screening for a compound which binds to NMDA ε4(NR2D), the method comprising the following steps (a) to (c):
(a) contacting NMDA ε4(NR2D) receptor protein with a test compound;
(b) detecting the binding of the test compound to NMDA ε4(NR2D) receptor protein; and
(c) selecting a test compound that binds with the NMDA ε4(NR2D) receptor protein.

6. A method of screening for a compound which binds to NMDA ε4(NR2D), the method comprising the following steps (a) to (c):
(a) contacting a test compound with a cell that expresses an NMDA ε4(NR2D) receptor gene in the presence of a ligand of the NMDA ε4(NR2D) receptor protein;
(b) detecting the activation of the NMDA ε4(NR2D) receptor; and
(c) selecting a compound for inhibiting the activation of the NMDA ε4(NR2D) receptor by comparison to the activation detected in the absence of the test compound.

7. A method of screening for a compound which binds to NMDA ε4(NR2D), the method comprising the following steps (a) to (c):
(a) contacting a test compound with a cell that expresses an NMDA ε4(NR2D) receptor gene;
(b) measuring the expression level of the NMDA ε4(NR2D) receptor gene; and
(b) selecting a compound that decreases the expression level in comparison to the level detected in the absence of the test compound.

8. A method of screening for a compound which binds to NMDA ε4(NR2D), the method comprising the following steps (a) to (c):
(a) contacting a test compound with a cell or cell extract containing DNA where the transcriptional control region of the NMDA ε4(NR2D) receptor gene is functionally linked to a reporter gene;
(b) measuring the expression level of the reporter gene; and
(c) selecting a compound that decreases the expression level of the reporter gene measured in step (b) above by comparison to the measurement conducted in the absence of a test compound.

9. A method for the determination of mechanical allodynia comprising;
detecting abnormality of DNA in an NMDA ε4(NR2D) receptor gene or the control region of the gene.

10. A method for the determination of mechanical allodynia comprising a step of detecting the expression of an NMDA ε4(NR2D) receptor gene or the molecular weight of the expressed gene product.

11. A test agent for use in the determination of mechanical allodynia comprising a nucleic acid which hybridizes to an NMDA ε4(NR2D) receptor gene or the control region of the gene and contains at least the strand length of 15 nucleotides.

12. A test agent for use in the determination of mechanical allodynia comprising an antibody that binds with an NMDA ε4(NR2D) receptor protein.
